(19) United States
(12) Patent Application Publication
(10) Pub. No.: US 2021/0008275 A1
(43) Pub. Date: Jan. 14, 2021

(54) PREFILLED SYRINGE, LIQUID MEDICINE ADMINISTRATION SYSTEM, AND SYRINGE PUMP

(71) Applicant: TERUMO KABUSHIKI KAISHA, Tokyo (JP)

(72) Inventors: Yuji OKUDA, Fujinomiya-shi (JP); Shota SEKIGUCHI, Ashigarakami-gun (JP)

(73) Assignee: TERUMO KABUSHIKI KAISHA, Tokyo (JP)

(21) Appl. No.: 17/037,212

(22) Filed: Sep. 29, 2020

Related U.S. Application Data
(63) Continuation of application No. PCT/JP2019/013334, filed on Mar. 27, 2019.

(30) Foreign Application Priority Data
Mar. 29, 2018 (JP) 2018-066113

(51) Int. Cl. A61M 5/145; A61M 5/32; A61M 5/31

(52) U.S. Cl. CPC A61M 5/1452; A61M 5/3202; A61M 5/3135; A61M 2205/6054; A61M 2205/52; A61M 2005/3131; A61M 2205/505; A61M 2205/3592; A61M 2205/6063

(57) ABSTRACT

A prefilled syringe is disclosed, which includes: a liquid medicine; a barrel including a cylindrical body section containing the liquid medicine, and a nozzle section provided on a distal end side of the body section that discharges the liquid medicine; a cap sealing a distal end opening; a gasket sliding on an inner circumferential surface of the body section; a plunger that can be mounted to the gasket; and an RFID tag having a fixed location with respect to the outer circumferential surface and has an antenna and a memory, the antenna being structured from an antenna wire wound in a rectangle, an outer diameter of the body section being 14 mm to 33 mm, the maximum circumferential length of the antenna is 9 mm to 25 mm, and the circumferential length of the outer circumferential surface is 2.0 times to 7.0 times the maximum circumferential length of the antenna.

### CROSS-REFERENCES TO RELATED APPLICATIONS

This application is a continuation of International Application No. PCT/JP2019/013334 filed on Mar. 27, 2019, which claims priority to Japanese Application No. 2018-066113 filed on Mar. 29, 2018, the entire content of both of which is incorporated herein by reference.

### FIELD OF THE INVENTION

The present disclosure generally relates to a prefilled syringe, a liquid medicine administration system, and a syringe pump.

### BACKGROUND DISCUSSION

When a medicine such as an intravenous anesthetic is delivered into the patient's body in a medical setting such as an operating room or an intensive care unit, it is necessary to deliver the medicine over a relatively long period of time while adjusting a flow rate (hereinafter also simply referred to as a “liquid delivery amount”) of the medicine to be delivered according to a technique to be applied, patient's condition, and the like. As a device for accurately delivering a medicine over the relatively long period of time with a set liquid delivery amount, liquid medicine administration devices, such as a syringe pump are known, which are capable of delivering a liquid medicine containing a medicine using a prefilled syringe that contains the liquid medicine in a barrel.

Japanese Patent Application Publication No. 2015-217176A discloses a liquid medicine injection device as a liquid medicine administration device of this type. Japanese Patent Application Publication No. 2015-217176 A also discloses a syringe which is to be mounted on the liquid medicine injection device and which has a radio frequency identification (RFID) tag attached to the syringe, the RFID tag having various kinds of data recorded on the RFID tag. Further, the liquid medicine injection device disclosed in Japanese Patent Application Publication No. 2015-217176 A has an RFID module including a reader that acquires various kinds of recorded data from the RFID tag of the syringe.

When the liquid medicine injection device disclosed in Japanese Patent Application Publication No. 2015-217176 A is used, the syringe is mounted on a base part of the liquid medicine injection device in a medical setting. When the syringe is mounted, it is necessary to bring the RFID tag close to the reader of the liquid medicine injection device so that the reader of the liquid medicine injection device can acquire the data of the RFID tag of the syringe. Therefore, it is necessary to mount the cylindrical syringe on the base part of the liquid medicine injection device while the RFID tag of the syringe is aligned with the reader of the liquid medicine injection device. This work can be complicated for the medical staff. Further, when the positional alignment described above is performed by rotating the syringe about the axis of the syringe with the syringe being mounted on the base part, the RFID tag may be damaged due to a sliding movement between the RFID tag fixed on the outer circumferential surface of the syringe and the base part of the liquid medicine injection device.

Further, when the RFID tag is attached to the outer circumferential surface of the syringe, the attached RFID tag may be deformed, because the outer circumferential surface of the syringe is curved. In addition, due to the deformation, the RFID tag may be detached.

### SUMMARY

A prefilled syringe is disclosed that includes an RFID tag from which data can be rather easily read by a syringe pump and which is less likely to be detached, and a liquid medicine administration system. Another aspect of the present disclosure is to provide a syringe pump adapted to the prefilled syringe.

In accordance with an aspect, a prefilled syringe is disclosed, which includes: a liquid medicine; a barrel including a body section that is cylindrical and that contains the liquid medicine, and a nozzle section that is provided on a distal end side of the body section and configured to discharge the liquid medicine; a cap configured to seal a distal end opening provided on a distal end section of the nozzle section; a gasket configured to slide on an inner circumferential surface of the body section; a plunger configured to be attachable to the gasket; and an RFID tag that has a fixed location with respect to an outer circumferential surface of the body section and has an antenna for communication and a memory, the antenna being structured from an antenna wire that is wound in a rectangle, wherein an outer diameter of the body section of the barrel is 14 mm to 33 mm, a maximum circumferential length of the antenna of the RFID tag along a circumferential direction of the body section of the barrel is 9 mm to 25 mm, and a circumferential length of the outer circumferential surface of the body section of the barrel is 2.0 times to 7.0 times the maximum circumferential length of the antenna of the RFID tag.

In accordance with an exemplary embodiment, a maximum axial length of the RFID tag along an axial direction of the body section of the barrel is 8 mm to 25 mm.

In accordance with another exemplary embodiment, the barrel has a flange that protrudes from a proximal end section of the body section, the body section has, near the flange, a clamp contact portion where a clamp section of a syringe pump that drives the plunger contacts, and the RFID tag is disposed distal to the clamp contact portion in the axial direction of the body section.

In accordance with an exemplary embodiment, a distance between a proximal end of the RFID tag and a distal end of the flange of the barrel is 15 mm to 40 mm in the axial direction of the body section.

In accordance with another exemplary embodiment, the RFID tag is located proximal to the distal end of the gasket in the axial direction of the body section.

In accordance with an exemplary embodiment, the prefilled syringe further includes an information label attached to the body section of the barrel, wherein the RFID tag is attached on an inner surface or an outer surface of the information label.

In accordance with another exemplary embodiment, the prefilled syringe further includes an information label attached to the body section of the barrel so as to cover an outer surface of the RFID tag.

In accordance with an exemplary embodiment, the information label has a scale indicating an amount of the liquid medicine in the body section, the scale being provided along an axial direction of the body section of the barrel, and at least a part of the RFID tag is located at a position overlapping with at least a part of the scale in the axial direction of the body section of the barrel and displaced in the circumferential direction of the body section of the barrel.

In accordance with another exemplary embodiment, the scale includes a plurality of scales, and the RFID tag is disposed between at least two of the scales.

In accordance with an exemplary embodiment, the at least two scales are symmetrical with respect to a central axis of the body section of the barrel.

In accordance with another aspect, a liquid medicine administration system is disclosed, which includes a syringe pump, and a prefilled syringe mounted on the syringe pump, the prefilled syringe including a liquid medicine, a barrel including a body section that is cylindrical and that contains the liquid medicine, and a nozzle section that is provided on a distal end side of the body section and configured to discharge the liquid medicine, a cap configured to seal a distal end opening provided on a distal end section of the nozzle section, a gasket configured to slide on an inner circumferential surface of the body section, a plunger mountable to the gasket, and an RFID tag that has a fixed location with respect to an outer circumferential surface of the body section and has an antenna for communication and a memory, the antenna being structured from an antenna wire that is wound in a rectangle, the syringe pump including a syringe plunger driving section configured to drive the plunger of the prefilled syringe in a distal direction toward the distal end of the body section, a main body provided with a reader and a control unit configured to control the syringe plunger driving section, the reader configured to read a data set stored in the memory of the RFID tag of the prefilled syringe, a supporting section configured to support the outer circumferential surface of the body section of the prefilled syringe in a direction perpendicular to an axis of the body section, and a clamp section configured to face the supporting section and to clamp the body section with the supporting section, wherein an outer diameter of the body section of the barrel is 14 mm to 33 mm, a maximum circumferential length of the antenna of the RFID tag along a circumferential direction of the body section of the barrel is 9 mm to 25 mm, and a circumferential length of the outer circumferential surface of the body section of the barrel is 2.0 times to 7.0 times the maximum circumferential length of the antenna of the RFID tag.

In accordance with an exemplary embodiment, when the clamp section clamps the prefilled syringe between the supporting section and the clamp section, a region clamped by the clamp section in the outer circumferential surface of the body section is different from a region where the RFID tag is attached.

In accordance with another exemplary embodiment, the barrel has a flange that protrudes from a proximal end section of the body section, a distance between a proximal end of the RFID tag and a distal end of the flange of the barrel is 15 mm to 40 mm in the axial direction of the body section, and the clamp section comes in contact with a vicinity of the flange of the body section, and clamps the prefilled syringe with the supporting section.

In accordance with an exemplary embodiment, the main body of the syringe pump has a first region facing the clamp section and a second region that is adjacent to the first region and is located on a side opposite to the syringe plunger driving section in the axial direction of the body section, the reader includes a reader antenna that communicates with the antenna of the RFID tag and that is structured from a reader antenna wire, and at least a part of the reader antenna is located in the second region.

In accordance with another exemplary embodiment, a centerline of the reader antenna perpendicular to the axial direction of the body section of the barrel is located in the second region in a front view of the supporting section, and the centerline of the reader antenna overlaps with the antenna of the RFID tag in the front view of the supporting section.

In accordance with an aspect, a liquid medicine administration system is disclosed, which includes a syringe pump to which a prefilled syringe is mounted, the prefilled syringe including a liquid medicine, a barrel including a body section that is cylindrical and that contains the liquid medicine, and a nozzle section that is provided on a distal end side of the body section and configured to discharge the liquid medicine, a cap configured to seal a distal end opening provided on a distal end section of the nozzle section, a gasket configured to slide on an inner circumferential surface of the body section, a plunger configured mountable to the gasket, and an RFID tag that has a fixed location with respect to an outer circumferential surface of the body section and has an antenna for communication and a memory, the antenna being structured from an antenna wire that is wound in a rectangle, the syringe pump including: a syringe plunger driving section configured to drive the plunger of the prefilled syringe in a distal direction toward the distal end of the body section; a main body provided with a reader and a control unit configured to control the syringe plunger driving section, the reader configured to read a data set stored in the memory of the RFID tag of the prefilled syringe; a supporting section configured to support the outer circumferential surface of the body section of the prefilled syringe in a direction perpendicular to an axis of the body section; and a clamp section configured to face the supporting section and to clamp the body section of the prefilled syringe with the supporting section, wherein the main body of the syringe pump has a first region facing the clamp section and a second region that is adjacent to the first region and is located on a side opposite to the syringe plunger driving section in an axial direction of the body section, the reader includes a reader antenna configured to communicate with the antenna of the RFID tag and that is structured from a reader antenna wire, and at least a part of the reader antenna is located in the second region.

In accordance with an exemplary embodiment, a centerline of the reader antenna perpendicular to the axial direction of the body section of the barrel is located in the second region in a front view of the supporting section.

The present disclosure can provide a prefilled syringe including an RFID tag from which data is rather easily read by a syringe pump and which is less likely to be detached, and a liquid medicine administration system. Moreover, the present disclosure can provide a syringe pump adapted to the prefilled syringe.

### BRIEF DESCRIPTION OF THE DRAWINGS

FIG. 1 is a perspective view showing a liquid medicine administration system including a prefilled syringe and a syringe pump in accordance with an exemplary embodiment.

FIG. 2 is a perspective view showing the prefilled syringe shown in FIG. 1.

FIG. 3 is a view showing an information label to be attached to the prefilled syringe shown in FIG. 2.

FIG. 4 is a block diagram showing an RFID tag attached to the prefilled syringe and a reader of the syringe pump.

FIG. 5 is a diagram showing an example of a method for obtaining a prefilled syringe having an information label attached to the prefilled syringe.

FIG. 6 is an enlarged front view in which a part of the front view of a main body of the syringe pump shown in FIG. 1 is enlarged.

FIG. 7 is a sectional view of the prefilled syringe and the syringe pump shown in FIG. 1.

FIG. 8 is a perspective view showing a prefilled syringe according to another exemplary embodiment.

FIG. 9 is a table (Table 1) illustrating six prefilled syringes 1 to 6 having different outer diameters of body sections of barrels were prepared and evaluated in accordance with an exemplary embodiment.

### DETAILED DESCRIPTION

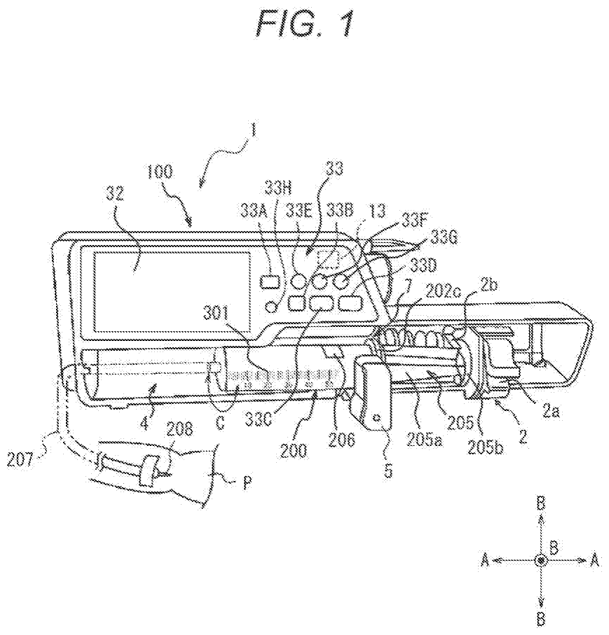

Set forth below with reference to the accompanying drawings is a detailed description of embodiments of a prefilled syringe, a liquid medicine administration system, and a syringe pump representing examples of the inventive prefilled syringe, liquid medicine administration system and syringe pump disclosed here. Hereinafter, embodiments of a prefilled syringe, a liquid medicine administration system, and a syringe pump according to the present disclosure will be described with reference to FIGS. 1 to 8. In the drawings, same members and parts are denoted by the same reference numerals.

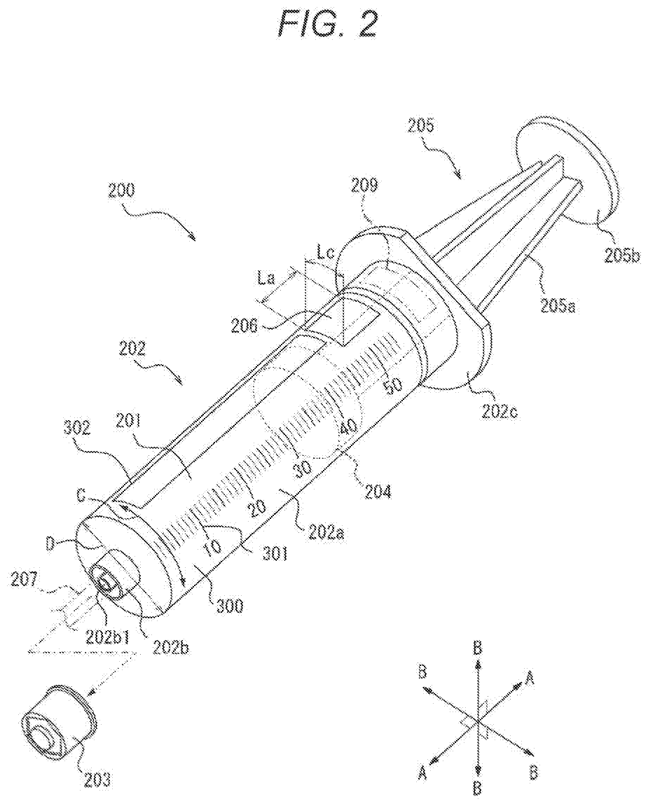

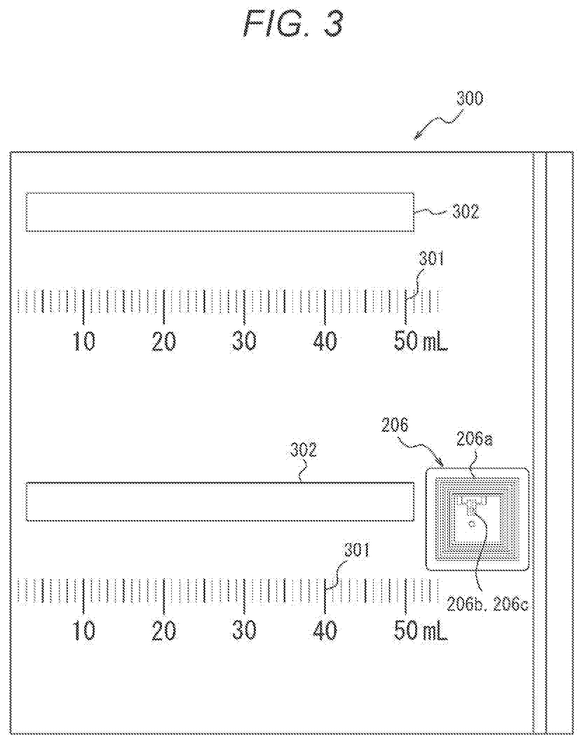

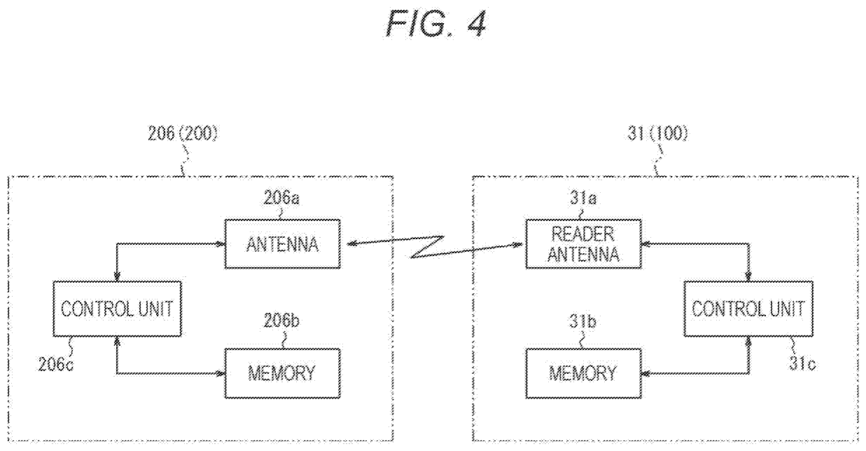

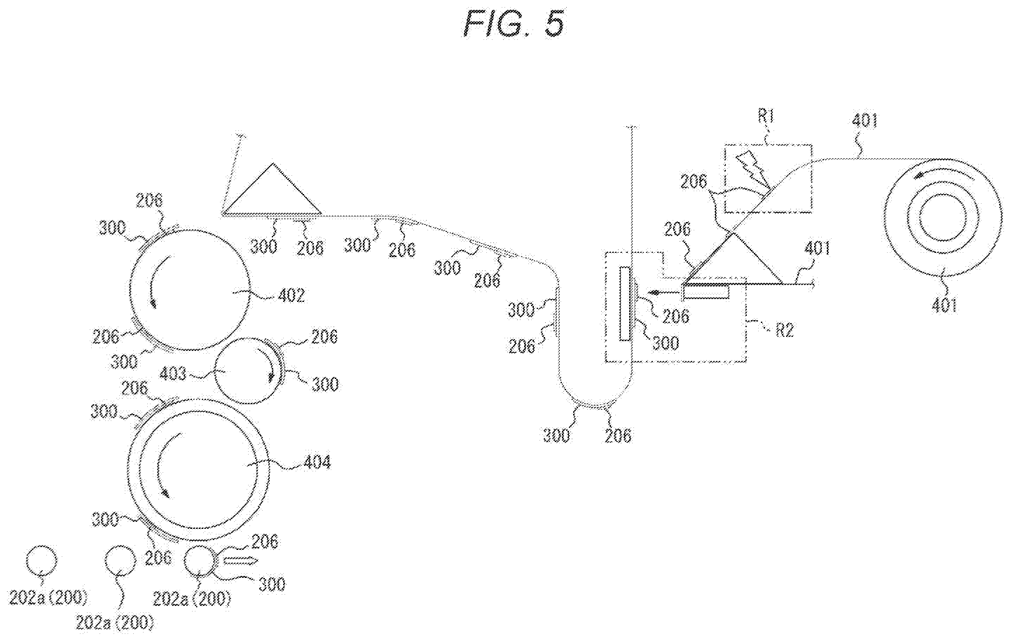

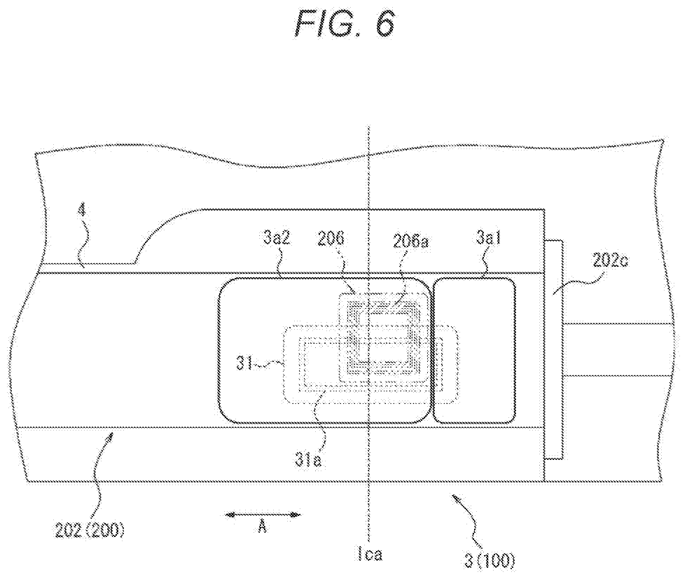

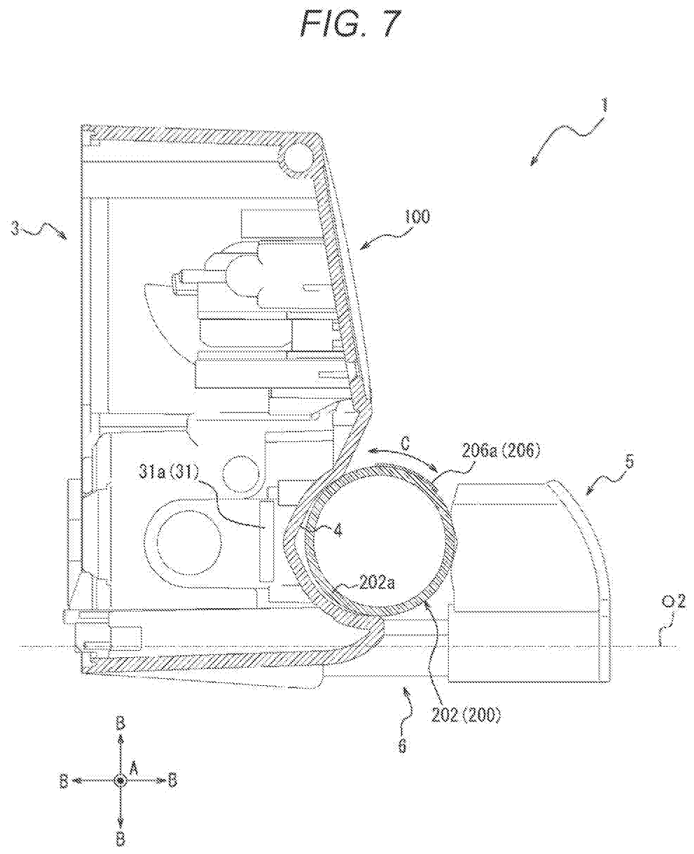

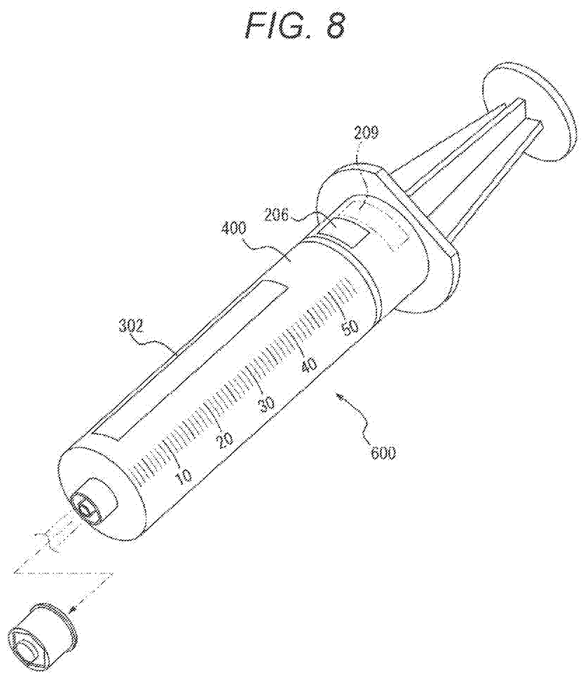

FIG. 1 is a perspective view showing a liquid medicine administration system 1 including a prefilled syringe 200 and a syringe pump 100 according to an exemplary embodiment. FIG. 2 is a perspective view showing the prefilled syringe 200 shown in FIG. 1. FIG. 3 is a view showing an information label 300 to be attached to the prefilled syringe 200 shown in FIG. 2. FIG. 4 is a block diagram showing an RFID tag 206 attached to the prefilled syringe 200 and a reader 31 of the syringe pump 100. FIG. 5 is a diagram showing an example of a method for obtaining the prefilled syringe 200 having the information label 300 attached to the prefilled syringe 200. FIG. 6 is an enlarged front view in which a part of the front view of a main body 3 of the syringe pump 100 shown in FIG. 1 is enlarged. FIG. 7 is a sectional view of the prefilled syringe 200 and the syringe pump 100 shown in FIG. 1. More specifically, FIG. 7 is a sectional view showing a cross section perpendicular to the axial direction of the prefilled syringe 200 in a state where the prefilled syringe 200 is mounted on the syringe pump 100. FIG. 8 is a perspective view showing a prefilled syringe 600 according to another exemplary embodiment.

As shown in FIG. 1, the liquid medicine administration system 1 according to the present exemplary embodiment includes the syringe pump 100 and the prefilled syringe 200 that can be mounted to the syringe pump 100. Each component will be described below.

### Prefilled Syringe 200

First, the prefilled syringe 200 will be described in detail. As shown in FIG. 2, the prefilled syringe 200 includes a liquid medicine 201, a barrel 202, a cap 203, a gasket 204, a plunger 205, and an RFID tag 206.

The liquid medicine 201 can be, for example, an anticancer agent, an anesthetic, a chemotherapeutic agent, a blood transfusion, a nutrient, or the like. The liquid medicine 201 is contained in the barrel 202. The liquid medicine 201 may be, for example, injected (i.e., microinjected) into the patient's body as described later.

The barrel 202 includes a cylindrical body section 202a, a nozzle section 202b, and a flange 202c.

The body section 202a contains the liquid medicine 201. An outer diameter D of the body section 202a can be, for example, 14 mm to 33 mm. Further, the circumferential length of the outer circumferential surface of the body section 202a is 2.0 times to 7.0 times the maximum circumferential length Lc of a later-described antenna of the RFID tag 206.

The nozzle section 202b is provided on the distal end side of the body section 202a. A distal end opening 202b1 is provided at a distal end section of the nozzle section 202b. The liquid medicine 201 is expelled from the distal end opening 202b1. A tube 207 (indicated by a chain double-dashed line in FIGS. 1 and 2) can be connected to the nozzle section 202b.

The flange 202c protrudes outward from the proximal end section of the body section 202a in the radial direction B of the body section 202a.

The cap 203 can seal the distal end opening 202b1 provided at the distal end section of the nozzle section 202b. The cap 203 can be attached to the nozzle section 202b, and FIG. 2 shows a state in which the cap 203 is removed from the nozzle section 202b and the distal end opening 202b1 is opened.

The gasket 204 slides on the inner circumferential surface of the body section 202a. The gasket 204 in the present exemplary embodiment is a tubular body having a closed distal end and an open proximal end. The gasket 204 constitutes a liquid medicine containing section in which the liquid medicine 201 is contained in the barrel 202. In the present exemplary embodiment, an amount of the liquid medicine contained within the liquid medicine containing section can be, for example, 5 ml to 50 ml. Note that, in general, an amount (ml) of liquid medicine contained within the liquid medicine containing section is associated with the outer diameter D (mm) of the prefilled syringe 200, and is specified in, for example, ISO 11040-6 (Prefilled syringes-Part 6: Plastic barrels for injectables). In the present exemplary embodiment, when a prefilled syringe, for example, containing 5 ml of liquid medicine is used, the outer diameter D is 14 mm. In addition, when the amount of the liquid medicine contained within the liquid medicine containing section, for example, is 10 ml, the outer diameter D is 17 mm. When the amount of the liquid medicine contained within the liquid medicine containing section, for example, is 20 ml, the outer diameter D is 22 mm. Further, when the amount of the liquid medicine contained within the liquid medicine containing section, for example, is 50 ml, the outer diameter D is 33 mm.

The plunger 205 can be mounted to the gasket 204. The plunger 205 includes a plunger body 205a and a flange 205b. In the present exemplary embodiment, the distal end section of the plunger body 205a can be fixed to the gasket 204 while being inserted from the proximal end side of the gasket 204. The plunger body 205a and the gasket 204 can be fixed to each other by, for example, thread connection. The plunger body 205a is inserted into the body section 202a and is movable in the axial direction A of the body section 202a within the body section 202a. The flange 205b protrudes outward from the plunger body 205a in the radial direction B on the proximal end section of the plunger 205.

In the present exemplary embodiment, the RFID tag 206 is attached to the outer surface of the information label 300 described later. That is, the RFID tag 206 in the present exemplary embodiment is attached to the outer circumferential surface of the body section 202a via the information label 300. In another exemplary embodiment, the information label 300 may be attached to the body section 202a of the barrel 202 so as to cover the outer surface of the RFID tag 206.

In accordance with an exemplary embodiment, the RFID tag 206 is not attached to the body section 202a over the entire circumference in the circumferential direction C, but is attached only to a part of the body section 202a in the circumferential direction C.

In the present exemplary embodiment, the maximum axial length La of the RFID tag 206 along the axial direction A of the body section 202a of the barrel 202, for example, is 8 mm to 25 mm. In the present exemplary embodiment, the proximal end of the RFID tag 206 is located distant from the distal end of the flange 202c of the barrel 202, for example, by 15 mm to 40 mm (for example, 24.8 mm) in a distal direction toward the distal end of the body section 202a in the axial direction A. In the present exemplary embodiment, the RFID tag 206 is located proximal to the distal end of the gasket 204 in the axial direction A of the body section 202a. Specifically, the RFID tag 206 in the present exemplary embodiment is located proximal to the distal end of the gasket 204 of the unused prefilled syringe 200 in the axial direction A of the body section 202a. In other words, the RFID tag 206 in the present embodiment is not located distal to the distal end of the gasket 204 in the axial direction A of the body section 202a. As shown in FIG. 1, in the present embodiment, a portion (hereinafter referred to as a clamp contact portion 209) clamped by a clamp section 5 of the syringe pump 100 in the outer circumferential surface of the prefilled syringe 200 is located proximal to the portion where the RFID tag 206 is attached.

For example, the RFID tag 206 can be formed into a rectangle, for example, having a circumferential length Lc of 18 mm and an axial length La of 18 mm in a state where the position is fixed with respect to the outer circumferential surface of the body section 202a. Alternatively, the RFID tag 206 may be formed into a rectangle, for example, having a circumferential length Lc of 25 mm and an axial length La of 25 mm in the same state (i.e., a state where the position of the RFID tag 206 is fixed with respect to the outer circumferential surface of the body section 202a of the barrel 202). The RFID tag 206 may also be formed into a rectangle, for example, having a circumferential length Lc of 12 mm and an axial length La of 9 mm in the same state.

As shown in FIGS. 3 and 4, the RFID tag 206 has an antenna 206a for communication, a memory 206b, and a control unit 206c.

As shown in FIG. 3, the antenna 206a of the RFID tag 206 is structured from an antenna wire wound in a rectangle. Although the area on the body section 202a where the RFID tag 206 can be installed is limited as shown in FIG. 2, a loop area formed by the antenna wire can be rather easily ensured, for example, by winding the antenna wire in a rectangle, as compared with a configuration in which the antenna wire, for example, is wound in a circle. Thus, even if the RFID tag 206 of the prefilled syringe 200 and the reader 31 of the syringe pump 100 are not aligned with relatively high precision when the prefilled syringe 200 is mounted on a supporting section 4 of the syringe pump 100, the RFID tag 206 can communicate with the reader 31, for example, as shown in FIGS. 1 and 7.

The antenna 206a of the RFID tag 206 performs communication by wireless communication, for example, having a relatively short working distance such as near field communication (NFC).

While the RFID tag 206 has a fixed location with respect to the outer circumferential surface of the body section 202a of the barrel 202, the maximum circumferential length Lc of the antenna 206a of the RFID tag 206 along the circumferential direction C of the body section 202a of the barrel 202, for example, can be 9 mm to 25 mm. For example, the outer perimeter of the antenna 206a can be formed into a rectangle, for example, having a circumferential length of 15 mm and an axial length of 15 mm in the abovementioned state. Alternatively, the outer perimeter of the antenna 206a may be formed, for example, into a rectangle having a circumferential length of 25 mm and an axial length of 25 mm in the abovementioned state.

The control unit 206c of the RFID tag 206 can read data from the memory 206b and cause the antenna 206a to transmit the data. In the wireless communication between the antenna 206a of the RFID tag 206 and a reader antenna 31a of the reader 31 of the syringe pump 100, a communicable distance may be relatively short (for example, within 35 mm). Therefore, when the RFID tag 206 of the prefilled syringe 200 is distant from the reader antenna 31a of the reader 31 by a predetermined distance or more, the reader antenna 31a of the reader 31 cannot communicate with the RFID tag 206 of the prefilled syringe 200. The memory 206b and the control unit 206c of the RFID tag 206 can be constituted by, for example, an integrated circuit (IC chip) including a non-volatile memory.

As shown in FIG. 4, the antenna 206a of the RFID tag 206 receives an electromagnetic wave transmitted from the reader antenna 31a of the reader 31 of the syringe pump 100. The operating power of the RFID tag 206 can be obtained from this electromagnetic wave. The control unit 206c reads the data in the memory 206b of the RFID tag 206, and sends (transmits) the data to the reader antenna 31a of the reader 31 through the electromagnetic wave using the antenna 206a. The reader antenna 31a of the reader 31 receives the electromagnetic wave from the antenna 206a of the RFID tag 206. Then, the control unit 31c of the syringe pump 100 acquires the data stored in the memory 206b of the RFID tag 206 by extracting the data from the received electromagnetic wave, and stores the data in the storage unit 31b of the syringe pump 100.

The memory 206b of the RFID tag 206 stores, for example, various kinds of data regarding the prefilled syringe 200, such as the name of the liquid medicine 201, identification data for each prefilled syringe, dimensional data of the barrel 202, and dimensional data of the stroke of the plunger 205.

As shown in FIG. 2, the information label 300 is attached to the outer circumferential surface of the body section 202a of the barrel 202. FIG. 3 shows the information label 300 which is not yet attached to the outer circumferential surface of the body section 202a.

The information label 300 is provided with two scales 301 which extend along the axial direction A of the body section 202a when the information label 300 is attached to the outer circumferential surface of the body section 202a (for example, see FIG. 2). More specifically, the notches of the two scales 301 in the present embodiment are arranged along the axial direction A of the body section 202a when the information label 300 is attached to the outer circumferential surface of the body section 202a. The scales 301 indicate the amount of the liquid medicine 201 in the body section 202a when the information label 300 is attached to the outer circumferential surface of the body section 202a. In the present exemplary embodiment, when the information label 300 is attached to the outer circumferential surface of the body section 202a of the barrel 202 as shown in FIG. 2, the two scales 301 are symmetrical with respect to the central axis of the body section 202a.

As shown in FIG. 3, the RFID tag 206 is attached between the two scales 301 of the information label 300. In the present exemplary embodiment, the RFID tag 206 is attached to the outer surface of the information label 300. In another exemplary embodiment, the RFID tag 206 can be attached to the inner surface of the information label 300.

In the present exemplary embodiment, a part of the RFID tag 206 is located at a position overlapping with a part of the two scales 301 in the longitudinal direction of the information label 300 and displaced in the transverse direction of the information label 300. That is, when the information label 300 is attached to the outer circumferential surface of the body section 202a as shown in FIG. 2, a part of the RFID tag 206 is located at a position overlapping with a part of the scales 301 in the axial direction A of the body section 202a of the barrel 202 and displaced in the circumferential direction C of the body section 202a of the barrel 202.

The information label 300 can be provided with an information area 302 in which the name of the medicine or an amount of the liquid medicine is written, for example, in addition to the scales 301.

In accordance with an aspect, an example of a method for obtaining the prefilled syringe 200 to which the information label 300 is attached will be described with reference to FIG. 5. First, a roll-shaped mount 401 is pulled out. RFID tags 206 are mounted on the mount 401, for example, at regular intervals. Next, various kinds of information are written in the RFID tag 206 as indicated by an area R1. Thereafter, as indicated by an area R2, the RFID tag 206 is removed from the mount 401, and the removed RFID tag 206 is attached to the outer surface of the information label 300. Then, the orientation of the information label 300 is properly set on a drum 402. Then, it is checked, on a drum 403, whether the information written in the RFID tag 206 is correct or not. Finally, the information label 300 is attached to the outer circumferential surface of the body section 202a of the prefilled syringe 200 using a drum 404.

### Syringe Pump 100

Next, the syringe pump 100 will be described with reference to FIGS. 1, 6, and 7.

In accordance with an exemplary embodiment, the syringe pump 100 may be used in, for example, an intensive care unit. Further, the syringe pump 100 can be used when a liquid medicine such as an anticancer agent, an anesthetic, a chemotherapeutic agent, a blood transfusion, or a nutrient is microinjected to a patient P over a relatively long period of time with relative high accuracy.

Further, the syringe pump 100 according to the present embodiment can be mounted to and removed, for example, from a stand or the like, and can be used while being mounted on the stand or the like. In accordance with an exemplary embodiment, the syringe pump 100 is fixed such that the axial direction A of the body section 202a of the prefilled syringe 200 mounted on the syringe pump 100 coincides with the horizontal direction.

As shown in FIG. 1, the syringe pump 100 includes a syringe plunger driving section 2, a main body 3, a supporting section 4, and a clamp section 5.

The syringe plunger driving section 2 drives the plunger 205 of the prefilled syringe 200 in the distal direction toward the distal end of the body section 202a.

The syringe plunger driving section 2 in the present embodiment includes a pressing part 2a and a flange fixing part 2b.

The pressing part 2a is located proximal to the flange 205b of the plunger 205 of the mounted prefilled syringe 200 in the axial direction A. Then, when the pressing part 2a is moved toward the distal side in the axial direction A, the surface of the flange 205b on the proximal side in the axial direction A can be pressed toward the distal side in the axial direction A. Accordingly, the plunger 205 can be relatively moved to the distal side in the axial direction A with respect to the barrel 202 of the mounted prefilled syringe 200.

In accordance with an exemplary embodiment, the flange fixing part 2b attaches (i.e., fixes) the flange 205b of the plunger 205 to the pressing part 2a. Specifically, the flange fixing part 2b in the present embodiment is located on the distal side of the pressing part 2a in the axial direction A and is attached to the pressing part 2a. While the prefilled syringe 200 is mounted, the flange 205b of the plunger 205 is located between the pressing part 2a and the flange fixing part 2b. Thus, the plunger 205 is movable in the axial direction A with the movement of the syringe plunger driving section 2 in the axial direction A.

FIG. 6 is an enlarged front view in which a part of the front view of the main body 3 of the syringe pump 100 is enlarged. More specifically, FIG. 6 is an enlarged view of a position of the later-described supporting section 4 that receives the prefilled syringe 200 in the main body 3. Note that, for convenience of description, FIG. 6 shows the barrel 202 of the prefilled syringe 200 which is appropriately received by the supporting section 4 with the flange 202c being engaged with a flange receiving groove 7 (for example, see FIG. 1) of the main body 3. The main body 3 is provided with the reader 31 including the reader antenna 31a that receives a data set stored in the memory of the RFID tag 206 of the prefilled syringe 200, and a control unit 13 (for example, see FIG. 1) that controls the syringe plunger driving section 2. Specifically, the reader 31 and the control unit 13 in the present embodiment are arranged inside the main body 3. Note that the control unit 13 of the syringe pump 100 and the control unit 31c described above can be provided as separate components. Further, the control unit 31c and the control unit 13 may be integrated as the same component. In the present exemplary embodiment, the reader antenna 31a of the reader 31 is structured from a reader antenna wire. As shown in FIG. 6, the outer perimeter of the reader antenna 31a structured from the reader antenna wire is rectangular. In the present embodiment, a part of the reader antenna 31a of the reader 31 is displaced from a later-described first region 3a1 that faces the later-described clamp section 5 (for example, see FIG. 1) of the syringe pump 100 in the axial direction A in a front view (same as the front view of the main body 3 in FIG. 6) of the supporting section 4 of the syringe pump 100.

In the present exemplary embodiment, the main body 3 has the first region 3a1 that faces the clamp section 5, and a second region 3a2 that is adjacent to the first region 3a1 on the side opposite to the syringe plunger driving section 2 in the axial direction A of the body section 202a. Then, at least a part of the reader antenna 31a of the reader 31 is located in the second region 3a2. Further, in the present exemplary embodiment, in the front view of the supporting section 4 (the same as the front view of the main body 3 in the present exemplary embodiment), the centerline Ica of the reader antenna 31a, which is perpendicular to the axial direction A of the body section 202a of the barrel 202, is located in the second region 3a2. Furthermore, in the present exemplary embodiment, the centerline Ica of the reader antenna 31a overlaps with the antenna 206a of the RFID tag 206 in the front view of the supporting section 4. Note that, since the outer perimeter of the reader antenna 31a is rectangular in the present exemplary embodiment, the centerline Ica of the reader antenna 31a is a line passing through the centers of two sides along the axial direction A of the body section 202a of the barrel 202 among the sides defining the perimeter of the reader antenna 31a. In accordance with an aspect, the centerline Ica of the reader antenna 31a is a line perpendicular to the axial direction A of the body section 202a of the prefilled syringe 200 and bisecting the loop area of the reader antenna 31a. If the perimeter of the reader antenna is not rectangular, a line that is perpendicular to the axial direction of the body section of the prefilled syringe and that bisects the loop area of the reader antenna may be used as the centerline.

The reader antenna 31a of the reader 31 can emit an electromagnetic wave in a state where the prefilled syringe 200 is received by the later-described supporting section 4. The RFID tag 206 attached to the prefilled syringe 200 transmits data in response to the electromagnetic wave. The reader antenna 31a of the reader 31 can receive the data.

Further, as shown in FIG. 1, the main body 3 in the present embodiment includes a display unit 32 and an operation panel 33.

In accordance with an aspect, the display unit 32 is an image display device capable of color display. The display unit 32 can be composed of, for example, a color liquid crystal display device. The display unit 32 can display not only information in Japanese but also information in a plurality of foreign languages as needed. The display unit 32 in the present exemplary embodiment is provided above the later-described supporting section 4 in the front surface of the syringe pump 100. The display unit 32 may include an input device such as a touch sensor and may receive an input from the user.

The operation panel 33 is disposed above the supporting section 4 and, for example, on the right of the display unit 32 in the front surface of the syringe pump 100. The operation panel 33 includes a power switch 33A, an operation indicator 33H, and operation switches. FIG. 1 shows a fast delivery switch 33B, a start switch 33C, a stop switch 33D, a display changeover switch 33E, a return/mute switch 33F, and a confirmation switch 33G as an example of the operation switches.

The operation on the operation panel 33 described above is input to the control unit 13, and each section of the syringe pump 100 operates according to a command from the control unit 13.

Further, the main body 3 in the present exemplary embodiment includes inside various wires for electrically connecting the components of the syringe pump 100, various mechanisms for executing commands from the control unit, a communication unit capable of wireless or wired communication with external devices other than the RFID tag 206, a storage unit that stores various kinds of data and various programs necessary for the operation of the syringe pump 100, and the like.

As shown in FIG. 1, the supporting section 4 in the present exemplary embodiment is formed on the front surface of the main body 3. Further, the supporting section 4 in the present exemplary embodiment supports the outer circumferential surface of the body section 202a of the prefilled syringe 200 in a direction perpendicular to the central axis of the body section 202a. As shown in FIG. 7, the supporting section 4 in the present exemplary embodiment is constituted by a concave curved surface having a substantially semicircular cross section in order to receive the outer circumferential surface of the body section 202a. Further, the supporting section 4 in the present exemplary embodiment can receive a plurality of types of body sections 202a, for example, having different sizes such as outer diameters.

As shown in FIG. 1, the clamp section 5 faces the supporting section 4 formed on the main body 3 and clamps the body section 202a of the prefilled syringe 200 with the supporting section 4. In the present exemplary embodiment, the clamp contact portion 209 (for example, see FIG. 2) which is a portion of the outer circumferential surface of the body section 202a where the clamp section 5 clamps the prefilled syringe 200 is different from the portion where the RFID tag 206 is attached, and is located proximal to the portion where the RFID tag 206 is attached.

As shown in FIG. 7, the clamp section 5 is supported by a base 6 so as to be movable in the longitudinal direction of the base 6 (direction parallel to the central axis O2). Therefore, the distance between the clamp section 5 and the supporting section 4 provided on the front surface of the main body 3 can be changed by moving the clamp section 5 in the longitudinal direction of the base 6, which makes it possible to move the clamp section 5 between a clamping position and a non-clamping position.

The clamping position of the clamp section 5 indicates a position where the body section 202a of the prefilled syringe 200 is clamped between the clamp section 5 and the supporting section 4 as shown in FIG. 7. Further, the non-clamping position of the clamp section 5 indicates a position where the clamp section 5 is moved further away from the supporting section 4 with respect to the clamp position so as not to clamp the body section 202a with the supporting section 4.

In accordance with an exemplary embodiment, the clamp section 5 is constantly biased by a biasing member such as a spring member in a direction approaching the supporting section 4 in the longitudinal direction of the base 6. Therefore, when the clamp section 5 is moved from the clamping position to the non-clamping position, the clamp section 5 is moved against the biasing force of the biasing member.

Further, the clamp section 5 is supported by the base 6 so as to be movable not only in the longitudinal direction of the base 6 but also in the circumferential direction around the central axis O2 of the base 6. As a result, the position of the base 6 in the longitudinal direction can be fixed so that the clamp section 5 does not move in the longitudinal direction of the base 6. Specifically, the clamp section 5 is rotatable in the circumferential direction around the central axis O2 of the base 6 between a position facing the supporting section 4 and a position not facing the supporting section 4. This rotation operation of the clamp section 5 is disabled when the clamp section 5 is at the clamping position, and is enabled when the clamp section 5 is at the non-clamping position. The clamp section 5 can be set to have an angle by which the clamp section 5 does not face the supporting section 4, and in this state, the prefilled syringe 200 can be rather easily placed on the receiving surface of the supporting section 4.

The prefilled syringe 200 operates as follows in the mounted state (for example, see FIG. 1) mounted on the syringe pump 100. The plunger 205 is pressed toward the distal side in the axial direction A by the syringe pump 100. As a result, the gasket 204 connected to the plunger 205 slides toward the distal side in the axial direction A within the body section 202a of the barrel 202.

When the gasket 204 slides toward the distal side in the axial direction A within the body section 202a, the liquid medicine 201 in the body section 202a is compressed. The liquid medicine 201 is expelled through the nozzle section 202b of the body section 202a by the compressive force. When the syringe pump 100 is used, the tube 207 (for example, see FIG. 1) is connected to the distal end opening 202b1 of the nozzle section 202b of the prefilled syringe 200. Further, as shown in FIG. 1, an indwelling needle 208 to be indwelled in the patient P is connected to the distal end of the tube 207. Therefore, the liquid medicine 201 in the body section 202a can be delivered into the body of the patient P through the tube 207 and the indwelling needle 208.

FIG. 8 shows a prefilled syringe 600 according to another exemplary embodiment. Hereinafter, the prefilled syringe 600 will be described as compared with the prefilled syringe 200 shown in FIG. 2. The longitudinal length ratio of an information label 400 in FIG. 8 is smaller than the longitudinal length ratio of the information label 300 in FIG. 2. The longitudinal length ratio means the ratio of the total length of the information label in the axial direction A to the total length of the barrel in the axial direction A of the body section. The RFID tag 206 in FIG. 8 has a configuration same as the configuration shown in FIG. 2. However, the distance (for example, 24.8 mm) between the flange 202c and the RFID tag 206 in the axial direction A in FIG. 2 is greater than the distance (for example, 11 mm) between the flange 202c and the RFID tag 206 in the axial direction A in FIG. 6. Note that, in the present embodiment, the clamp contact portion 209 of the syringe pump 100 is also located proximal to the portion where the RFID tag 206 is attached in FIG. 8. The other configuration of the prefilled syringe 600 is the same as that of the prefilled syringe 200, and thus the description of the prefilled syringe 200 will be omitted.

In the prefilled syringe 200 or 600 or the liquid medicine administration system 1 according to the exemplary embodiment of the present disclosure, the outer diameter D of the body section 202a of the barrel 202 is 14 mm to 33 mm. Further, the maximum circumferential length Lc of the antenna of the RFID tag 206 along the circumferential direction C of the body section 202a of the barrel 202 is 9 mm to 25 mm. Furthermore, the circumferential length of the outer circumferential surface of the body section 202a is 2.0 times to 7.0 times the maximum circumferential length of the antenna of the RFID tag 206.

With these configurations, even if the RFID tag 206 of the prefilled syringe 200 and the reader of the syringe pump 100 are not aligned when the cylindrical prefilled syringe 200 or 600 is mounted on the supporting section 4 of the syringe pump 100, the RFID tag 206 can communicate with the reader 31. Further, since the positional alignment of the prefilled syringe 200 or 600 is unnecessary, it is possible to prevent the RFID tag 206 fixed to the outer circumferential surface of the prefilled syringe 200 or 600 from being damaged because of the sliding movement between the RFID tag 206 of the prefilled syringe 200 or 600 and the supporting section 4 of the syringe pump 100.

In addition, when the RFID tag is attached to the outer circumferential surface of the prefilled syringe, the attached RFID tag may be deformed and detached, because the outer circumferential surface of the prefilled syringe is curved. On the other hand, due to the configuration described above, detachment of the RFID tag 206 attached to the outer circumferential surface of the prefilled syringe 200 or 600 can be prevented.

In the prefilled syringe 200 or 600 according to the embodiment of the present disclosure, the maximum axial length La of the RFID tag 206 along the axial direction of the body section 202a of the barrel 202 is 8 mm to 25 mm. Due to the maximum axial length La of the RFID tag 206 being set to 8 mm or more, the RFID tag 206 can more reliably communicate with the reader of the syringe pump 100 when the cylindrical prefilled syringe 200 or 600 is mounted on the supporting section 4 of the syringe pump 100. Further, due to the maximum axial length La of the RFID tag 206 being set to 25 mm or less, it is possible to prevent the scale 301 from being difficult to see by the RFID tag 206, and to prevent the RFID tag 206 from deteriorating visibility of the liquid medicine 201 contained in the body section 202a.

In the prefilled syringe 200 or 600 according to the embodiment of the present disclosure, the barrel 202 has the flange 202c protruding from the proximal end section of the body section 202a, the body section 202a has, near the flange 202c, the clamp contact portion 209 where the clamp section 5 of the syringe pump 100 that drives the plunger 205 contacts, and the RFID tag 206 is located distal to the clamp contact portion 209 in the axial direction A of the body section 202a, which helps prevent the clamp section 5 of the syringe pump 100 from contacting the RFID tag 206, thereby being capable of preventing the RFID tag 206 from being damaged.

In the prefilled syringe 200 or 600 according to the embodiment of the present disclosure, the distance between the proximal end of the RFID tag 206 and the distal end of the flange 202c of the barrel 202 in the axial direction of the body section 202a, for example, is 15 mm to 40 mm. Due to the configuration in which the distance between the proximal end of the RFID tag 206 and the distal end of the flange 202c of the barrel 202 is set to 15 mm or more in the axial direction of the body section 202a, the contact between the clamp section 5 of the syringe pump 100 and the RFID tag 206 can be prevented to help prevent damage of the RFID tag 206. Further, due to the configuration in which the distance between the proximal end of the RFID tag 206 and the distal end of the flange 202c of the barrel 202 is set to 40 mm or less in the axial direction of the body section 202a, it is possible to prevent the scale 301 from being difficult to see and to help ensure visibility of the liquid medicine 201 contained in the body section 202a.

In the prefilled syringe 200 or 600 according to the embodiment of the present disclosure, the RFID tag 206 is located proximal to the distal end of the gasket 204 in the axial direction A of the body section 202a, which configuration can help prevent the scale 301 from being difficult to see, and can help ensure visibility of the liquid medicine 201 contained in the body section 202a.

The prefilled syringe 200 according to the embodiment of the present disclosure further includes the information label 300 attached to the body section 202a of the barrel 202. The RFID tag 206 can be attached to the inner surface or the outer surface of the information label 300. With these configurations of the RFID tag 206 being attached to the inner surface or the outer surface of the information label 300, when the prefilled syringe 200 is manufactured, the information label 300 and the RFID tag 206 can be attached to the barrel 202 at one time as shown in FIG. 5, whereby the production efficiency of the prefilled syringe 200 can be improved.

The prefilled syringe 200 may be configured such that the information label 300 is attached to the body section 202a of the barrel 202 so as to cover the outer surface of the RFID tag 206, which can help prevent the RFID tag 206 fixed to the outer circumferential surface of the body section 202a from being detached.

In the prefilled syringe 200 or 600 according to the embodiment of the present disclosure, the information label 300 has the scale 301 indicating an amount of the liquid medicine 201 in the body section 202a, the scale 301 being provided along the axial direction A of the body section 202a of the barrel 202, and at least a part of the RFID tag 206 is located at a position overlapping with at least a part of the scale 301 in the axial direction A of the body section 202a of the barrel 202 and displaced in the circumferential direction C of the body section 202a of the barrel 202, which helps make it possible to provide the scale 301 and the RFID tag 206 on the prefilled syringe 200 or 600 without increasing the axial length of the prefilled syringe 200 or 600.

In the prefilled syringe 200 or 600 according to the exemplary embodiment of the present disclosure, a plurality of scales 301 may be provided, and the RFID tag 206 is located between at least two scales 301. With this configuration of the plurality of scales 301 and the RFID tag 206 being located between at least two scales 206, when the prefilled syringe 200 or 600 is mounted on the syringe pump 100, at least one scale 301 can be exposed and visually recognized.

In the prefilled syringe 200 or 600 according to the exemplary embodiment of the present disclosure, at least two scales 301 may be symmetrical with respect to the central axis of the body section 202a of the barrel 202. With this configuration of the at least two scales 301 being symmetrical with respect to the central axis of the body section 202a of the barrel 200, at least one scale 301 can be exposed and visually recognized more reliably.

In the liquid medicine administration system 1 according to the exemplary embodiment of the present disclosure, the syringe pump 100 has the clamp section 5 that faces the supporting section 4 and clamps the body section 202a of the prefilled syringe 200 or 600 with the supporting section 4, and when the clamp section 5 clamps the prefilled syringe 200 or 600 between the supporting section 4 and the clamp section 5, the clamp contact portion 209 which is a region clamped by the clamp section 5 in the outer circumferential surface of the body section 202a is different from a region where the RFID tag 206 is attached, which helps prevent the clamp section 5 of the syringe pump 100 from contacting the RFID tag 206, thereby being capable of preventing the RFID tag 206 from being damaged.

In the liquid medicine administration system 1 according to the exemplary embodiment of the present disclosure, the barrel 202 has the flange 202c that protrudes from the proximal end section of the body section 202a, a distance between the proximal end of the RFID tag 206 and the distal end of the flange 202c of the barrel 202, for example, is 15 mm to 40 mm in the axial direction of the body section 202a, and the clamp section 5 comes in contact within a vicinity of the flange 202c of the body section 202a, and clamps the prefilled syringe 200 or 600 with the supporting section 4. Due to the configuration in which the distance between the proximal end of the RFID tag 206 and the distal end of the flange 202c of the barrel 202, for example, is set to 15 mm or more in the axial direction of the body section 202a, and the clamp section 5 of the syringe pump 100 comes in contact with the clamp contact portion 209 which is in the vicinity of the flange 202c of the body section 202a, the contact between the clamp section 5 and the RFID tag 206 can be prevented and corresponding damage to the RFID tag 206 can also be prevented. Further, due to the configuration in which the distance between the proximal end of the RFID tag 206 and the distal end of the flange 202c of the barrel 202, for example, is set to 40 mm or less in the axial direction of the body section 202a, it is possible to prevent the scale 301 from being difficult to see and can help ensure the visibility of the liquid medicine 201 contained in the body section 202a.

In the liquid medicine administration system 1 according to the exemplary embodiment of the present disclosure, the main body 3 of the syringe pump 100 has the first region 3a1 facing the clamp section 5 and the second region 3a2 that is adjacent to the first region 3a1 and is located on a side opposite to the syringe plunger driving section 2 in the axial direction A of the body section 202a, the reader 31 includes the reader antenna 31a that communicates with the antenna 206a of the RFID tag 206 and that is structured from a reader antenna wire, and at least a part of the reader antenna 31a is located in the second region 3a2. That is, at least a part of the reader antenna 31a is arranged in the second region 3a2, which is not the first region 3a1 facing the clamp section 5. With this configuration, the reader antenna 31a can reliably read the data set of the RFID tag 206. Note that the prefilled syringe 200 or 600 is mounted to the syringe pump 100 such that at least a part of the RFID tag 206 faces the second region 3a2.

In the liquid medicine administration system 1 according to the exemplary embodiment of the present disclosure, the centerline Ica of the reader antenna 31a perpendicular to the axial direction of the body section 202a of the barrel 202 is located in the second region 3a2 in a front view of the supporting section 4, and the centerline Ica of the reader antenna 31a overlaps with the antenna of the RFID tag 206 in the front view of the supporting section 4. Here, it is preferable that the position of the centerline Ica of the reader antenna 31a substantially coincides with the position where the magnetic flux density of the magnetic flux generated by the reader antenna 31a is the highest. Therefore, due to the configuration in which the centerline Ica of the reader antenna 31a overlaps with the antenna of the RFID tag 206, the reader 31 can more reliably read the data set of the RFID tag 206.

The syringe pump 100 according to the exemplary embodiment of the present disclosure includes the clamp section 5 that faces the supporting section 4 and clamps the body section 202a of the prefilled syringe 200 with the supporting section 4, the main body 3 of the syringe pump 100 has the first region 3a1 facing the clamp section 5 of the body section 202a and the second region 3a2 that is adjacent to the first region 3a1 and is located on a side opposite to the syringe plunger driving section 2 in the axial direction A of the body section 202a, the reader 31 includes the reader antenna 31a that communicates with the antenna of the RFID tag 206 and that is structured from a reader antenna wire, and at least a part of the reader antenna 31a is located in the second region 3a2. With this configuration, at least a part of the reader antenna 31a is arranged in the second region 3a2, which is not the first region 3a1 facing the clamp section 5, so that the reader antenna 31a can read the data set of the RFID tag 206. Further, when the prefilled syringe 200 is clamped by the supporting section 4 and the clamp section 5, the RFID tag 206 can be prevented from being damaged due to contact with the clamp section 5. Note that the prefilled syringe 200 or 600 is mounted to the syringe pump 100 such that at least a part of the RFID tag 206 faces the second region 3a2.

Further, in the syringe pump 100 according to the exemplary embodiment of the present disclosure, the centerline Ica of the reader antenna 31a, which is perpendicular to the body section 202a of the barrel 202, is located in the second region 3a2 in a front view of the supporting section 4, which allows the reader 31 to reliably read the data of the RFID tag 206 of the syringe pump 100.

### Examples

Next, prefilled syringes according to the exemplary embodiment of the present disclosure were manufactured and their performance was evaluated. This will be described below.

In this example, six prefilled syringes 1 to 6 having different outer diameters of body sections of barrels were prepared. The outer diameters of the body sections of the barrels of the prefilled syringes 1 to 6 were 11 mm, 14 mm, 17 mm, 22 mm, 33 mm, and 35.5 mm, respectively.

### Verification of Attachment of RFID Tag

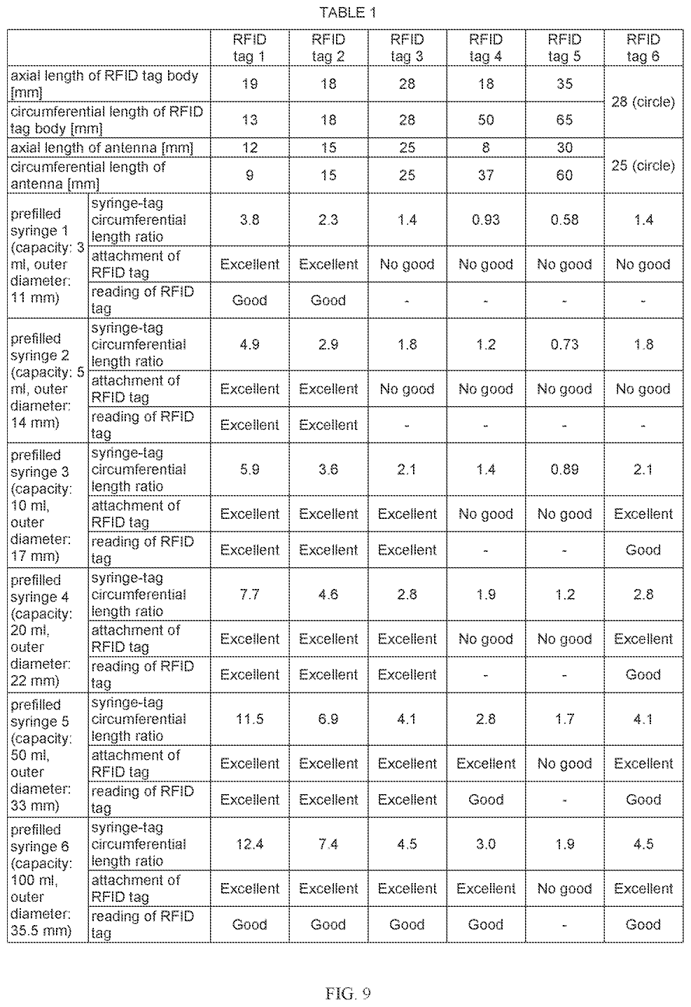

RFID tags 1 to 6 were attached to the outer circumferential surfaces of the body sections of the barrels of the prefilled syringes 1 to 6, and then, whether the RFID tags 1 to 6 were detached or not was checked. In each of the RFID tags 1 to 5, the shape of the tag body including an antenna, a memory, and a control unit and the outer perimeter of an antenna wire are rectangular. Further, the shape of a tag body of the RFID tag 6 is a circle with an outer diameter of 28 mm, and the outer perimeter of an antenna wire is a circle with an outer diameter of 25 mm. FIG. 9 (Table 1) shows the dimensions of the tag bodies of the RFID tags 1 to 6 and the dimensions of the antennas. Table 1 also shows values (indicated as “syringe-tag circumferential length ratios” in Table 1) each obtained by dividing the circumferential length of the outer circumferential surface of the body section of the barrel of prefilled syringe by the maximum circumferential length of the antenna of the RFID tag. In this example, the RFID tags 1 to 6 were attached to the outer circumferential surfaces of the body sections of the prefilled syringes 1 to 6, respectively, and then, it was checked whether the RFID tags 1 to 6 were detached. “0” in Table 1 shows the case where the RFID tag was not detached. “X” indicates that the RFID tag could not be attached due to its large size, or that the RFID tag was attached and then detached, in other words, the RFID tag could not be properly attached to the prefilled syringe.

### Verification Regarding Reading of RFID Tag

The RFID tags 1 to 6 were attached to the outer circumferential surfaces of the body sections of the barrels of the prefilled syringes 1 to 6, and each of the prefilled syringes 1 to 6 was mounted on the syringe pump 100 shown in FIG. 1. Here, the prefilled syringes 1 to 6 were mounted such that the attached RFID tags 1 to 6 were at positions farthest from the reader 31 of the syringe pump 100, that is, the RFID tag 206 faced the front side in FIG. 1. In such a state, it was checked multiple times whether the reader 31 of the syringe pump 100 could read the data of the RFID tags 1 to 6. “Excellent” in Table 1 indicates that the RFID tag data could be read in all cases. “Good” indicates that the RFID tag data could not be read in some cases. Furthermore, “-” indicates that the test was not performed because the RFID tag could not be properly attached to the outer circumferential surface of the body section of the barrel of the prefilled syringe.

As is clear from FIG. 9 (Table 1), in prefilled syringes each configured such that the antenna is structured from an antenna wire wound in a rectangle, the outer diameter of the body section of the barrel is 14 mm to 33 mm, the maximum circumferential length Lc of the antenna of the RFID tag along the circumferential direction of the body section a of the barrel is 9 mm to 25 mm, and the circumferential length of the outer circumferential surface of the body section is set to be 2.0 to 7.0 times the maximum circumferential length of the antenna of the RFID tag 206, the syringe pump more easily acquires data, and the attached RFID tag is less likely to be detached, as compared to the other prefilled syringes.

The detailed description above describes embodiments of a prefilled syringe, a liquid medicine administration system, and a syringe pump. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A prefilled syringe comprising:
a liquid medicine;
a barrel including a body section that is cylindrical and that contains the liquid medicine, and a nozzle section that is provided on a distal end side of the body section and configured to discharge the liquid medicine;
a cap configured to seal a distal end opening provided on a distal end section of the nozzle section;
a gasket configured to slide on an inner circumferential surface of the body section;
a plunger configured to be attachable to the gasket;
an RFID tag that has a fixed location with respect to an outer circumferential surface of the body section and has an antenna for communication and a memory, the antenna being structured from an antenna wire that is wound in a rectangle;
wherein an outer diameter of the body section of the barrel is 14 mm to 33 mm;
a maximum circumferential length of the antenna of the RFID tag along a circumferential direction of the body section of the barrel is 9 mm to 25 mm; and
a circumferential length of the outer circumferential surface of the body section of the barrel is 2.0 times to 7.0 times the maximum circumferential length of the antenna of the RFID tag.

2. The prefilled syringe according to claim 1, wherein a maximum axial length of the RFID tag along an axial direction of the body section of the barrel is 8 mm to 25 mm.

3. The prefilled syringe according to claim 2,
wherein the barrel has a flange that protrudes from a proximal end section of the body section;
the body section has, near the flange, a clamp contact portion configured to be in contact with a clamp section of a syringe pump that drives the plunger; and
the RFID tag is disposed distal to the clamp contact portion in the axial direction of the body section.

4. The prefilled syringe according to claim 2, wherein the RFID tag is located proximal to the distal end of the gasket in the axial direction of the body section.

5. The prefilled syringe according to claim 2, further comprising:
an information label attached to the body section of the barrel; and
wherein the RFID tag is attached to an inner surface or an outer surface of the information label.

6. The prefilled syringe according to claim 1,
wherein the barrel includes a flange that protrudes from a proximal end section of the body section;
the body section includes, near the flange, a clamp contact portion configured to be in contact with a clamp section of a syringe pump that drives the plunger; and
the RFID tag is disposed distal to the clamp contact portion in an axial direction of the body section.

7. The prefilled syringe according to claim 6, wherein a distance between a proximal end of the RFID tag and a distal end of the flange of the barrel is 15 mm to 40 mm in the axial direction of the body section.

8. The prefilled syringe according to claim 1, wherein the RFID tag is located proximal to the distal end of the gasket in the axial direction of the body section.

9. The prefilled syringe according to claim 1, further comprising:
an information label attached to the body section of the barrel; and
wherein the RFID tag is attached to an inner surface of the information label or an outer surface of the information label.

10. The prefilled syringe according to claim 1, further comprising:
an information label attached to the body section of the barrel, the information label configured to cover an outer surface of the RFID tag.

11. The prefilled syringe according to claim 9,
wherein the information label has a scale indicating an amount of the liquid medicine in the body section, the scale being provided along an axial direction of the body section of the barrel; and
at least a part of the RFID tag is located at a position overlapping with at least a part of the scale in the axial direction of the body section of the barrel and displaced in a circumferential direction of the body section of the barrel.

12. The prefilled syringe according to claim 11,
wherein the scale includes a plurality of scales; and
the RFID tag is disposed between at least two of the plurality of scales.

13. The prefilled syringe according to claim 12, wherein the at least two of the plurality scales are symmetrical with respect to a central axis of the body section of the barrel.

14. A liquid medicine administration system comprising:
a syringe pump;
a prefilled syringe configured to be attached to the syringe pump, the prefilled syringe comprising:
a liquid medicine;
a barrel including a body section that is cylindrical and that contains the liquid medicine, and a nozzle section that is provided on a distal end side of the body section and configured to discharge the liquid medicine;
a cap configured to seal a distal end opening provided on a distal end section of the nozzle section;
a gasket configured to slide on an inner circumferential surface of the body section;
a plunger configured to be attached to the gasket; and
an RFID tag that has a fixed location with respect to an outer circumferential surface of the body section and has an antenna for communication and a memory, the antenna being structured from an antenna wire that is wound in a rectangle;
the syringe pump comprising:
a syringe plunger driving section configured to drive the plunger of the prefilled syringe in a distal direction toward the distal end of the body section;
a main body provided with a reader and a control configured to control the syringe plunger driving section, the reader configured to read a data set stored in the memory of the RFID tag of the prefilled syringe;
a supporting section configured to support the outer circumferential surface of the body section of the prefilled syringe in a direction perpendicular to an axis of the body section; and
a clamp section configured to face the supporting section and to clamp the body section with the supporting section;
wherein an outer diameter of the body section of the barrel is 14 mm to 33 mm;
a maximum circumferential length of the antenna of the RFID tag along a circumferential direction of the body section of the barrel is 9 mm to 25 mm; and
a circumferential length of the outer circumferential surface of the body section of the barrel is 2.0 times to 7.0 times the maximum circumferential length of the antenna of the RFID tag.

15. The liquid medicine administration system according to claim 14, wherein, when the clamp section clamps the prefilled syringe between the supporting section and the clamp section, a region clamped by the clamp section in the outer circumferential surface of the body section is different from a region where the RFID tag is attached.

16. The liquid medicine administration system according to claim 15,
wherein the barrel has a flange that protrudes from a proximal end section of the body section;
a distance between a proximal end of the RFID tag and a distal end of the flange of the barrel is 15 mm to 40 mm in an axial direction of the body section, and
the clamp section comes in contact within a vicinity of the flange of the body section, and clamps the prefilled syringe with the supporting section.

17. The liquid medicine administration system according to claim 16,
wherein the main body of the syringe pump has a first region facing the clamp section and a second region that is adjacent to the first region and is located on a side opposite to the syringe plunger driving section in the axial direction of the body section;
the reader includes a reader antenna configured to communicate with the antenna of the RFID tag and that is structured from a reader antenna wire; and
at least a part of the reader antenna is located in the second region.

18. The liquid medicine administration system according to claim 17,
wherein a centerline of the reader antenna perpendicular to the axial direction of the body section of the barrel is located in the second region in a front view of the supporting section; and
the centerline of the reader antenna overlaps with the antenna of the RFID tag in the front view of the supporting section.

19. A liquid medicine administration system comprising:
a syringe pump to which a prefilled syringe is mounted, the prefilled syringe comprising:
a liquid medicine;
a barrel including a body section that is cylindrical and that contains the liquid medicine, and a nozzle section that is provided on a distal end side of the body section and configured to discharge the liquid medicine;
a cap configured to seal a distal end opening provided on a distal end section of the nozzle section;
a gasket configured to slide on an inner circumferential surface of the body section;
a plunger configured to be attachable to the gasket; and
an RFID tag that has a fixed location with respect to an outer circumferential surface of the body section and has an antenna for communication and a memory, the antenna being structured from an antenna wire that is wound in a rectangle; and
the syringe pump comprising:
a syringe plunger driving section configured to drive the plunger of the prefilled syringe in a distal direction toward the distal end of the body section;
a main body provided with a reader and a control unit configured to control the syringe plunger driving section, the reader configured to read a data set stored in the memory of the RFID tag of the prefilled syringe;
a supporting section configured to support the outer circumferential surface of the body section of the prefilled syringe in a direction perpendicular to an axis of the body section;
a clamp section configured to face the supporting section and to clamp the body section of the prefilled syringe with the supporting section;
wherein the main body of the syringe pump has a first region facing the clamp section and a second region that is adjacent to the first region and is located on a side opposite to the syringe plunger driving section in an axial direction of the body section;
the reader includes a reader antenna configured to communicate with the antenna of the RFID tag and that is structured from a reader antenna wire, and at least a part of the reader antenna is located in the second region.

20. The liquid medicine administration system according to claim 19, wherein a centerline of the reader antenna perpendicular to the axial direction of the body section of the barrel is located in the second region in a front view of the supporting section.
